(19) United States
(12) Patent Application Publication
(10) Pub. No.: US 2004/0249298 A1
(43) Pub. Date: Dec. 9, 2004

(54) Method and apparatus for determining heart rate

(76) Inventor: James R. Selevan, Laguna Beach, CA (US)

Correspondence Address: JAMES R. SELEVAN, 1661 Sunset Ridge Drive, Laguna Beach, CA, 92651, US

(21) Appl. No.: 10/859,789

(22) Filed: Jun. 3, 2004

Related U.S. Application Data
(60) Provisional application No. 60/475,241, filed on Jun. 3, 2003.

(51) Int. Cl. A61B 5/0402

(52) U.S. Cl. 600/528

(57) ABSTRACT

Devices and methods are disclosed for measuring the heart rate of an animal and more specifically, a human. Heart rate measurements are an integral part of many physical examinations. The devices and methods disclosed herein are useful in any field of medicine where a heart rate is measured. The devices utilize a standard medical stethoscope with a removably mounted timer attached thereto. The stethoscope is used by the medical caregiver to listen to cardiac sounds, and more specifically, the heart beats or arterial blood pulses in conjunction with a sphygmomanometer. A countdown timer is attached to the stethoscope between the “Y” and the auscultation head. The countdown timer is actuated by the medical caregiver and the number of cardiac beats are counted until the timer reaches zero. The timer provides an audible warning of timeout and optionally provides a visual indication of timeout. The proximity of the timer to the stethoscope obviates the need to stare at a watch or clock as it counts out the correct amount of seconds. The number of seconds in the countdown sequence is variable and determined from a series of choices provided to the caregiver.

### RELATED APPLICATIONS

This application claims priority benefit under 35 USC § 119(e) from U.S. Provisional Application No. 60/475,241, filed on Jun. 3, 2003, entitled “METHOD AND APPARATUS FOR DETERMINING HEART RATE”, the entirety of which is hereby incorporated herein by reference.

### FIELD OF THE INVENTION

The field of this invention is general medicine, family practice, pediatric medicine, emergency medicine, and cardiology. More specifically, the field relates to devices and methods for measuring the pulse rate or breathing rate of a human or other animal.

### BACKGROUND OF THE INVENTION

Measurement of heart rate is relatively common in medicine. Heart or pulse rate measurements are used to assist with determination of the physical health of a patient as well as to determine the extent of any pathologies. One way to measure heart rate is to feel for a pulse, usually done by pressing a finger against a specific location on the wrist or neck of a patient. The specific location preferred for pulse measurements is one where an arterial blood vessel runs close to the surface of the skin and is not overlaid by excessive fat, bone, or muscle. In another method, medical caregivers, including physicians, nurses, EMTs, paramedics, and the like, use a stethoscope to listen to the sounds of the heart or the sounds of blood moving through the arterial vasculature. In either method, the medical caregiver generally counts pulses for a period of time and performs a mathematical computation to determine the number of pulses that occur per minute. A heart rate is generally measured in units of beats per minute.

The use of manual stethoscopes to measure heart rate or breathing rate is unlikely to change in the foreseeable future. Electronic stethoscopes are not commonly used even though they are available and use of an electronic stethoscope requires visual observation of the digital output display to measure the heart or breathing rate. The use of a wristwatch or wall clock to measure a time interval increases the risk that an incorrect measurement will be made because of a misinterpretation of the time.

New devices and methods are needed to permit rapid and simple heart rate data acquisition during a prescribed time interval. These devices and methods preferably work without the need to visually observe a display to obtain time or rate information. In addition, improved devices and methods of are needed to reduce inaccuracies that occur when a time interval is misread by the person measuring the breathing rate, heart rate or pulse rate of a patient.

### SUMMARY OF THE INVENTION

This invention relates to improved devices and methods for measuring the heart or breathing rate of a patient. More generally, the invention relates to improved devices and methods for measuring the heart or breathing rate of any animal. The heart rate is generally the same as the pulse rate of an animal so the invention is applicable to measurement of the pulse rate of an animal or human. The invention is also suitable for measurement of the pulmonary or breathing rate of a human or other animal.

The invention comprises a timer that is attached to a stethoscope. The timer further comprises an audio output system to indicate that a countdown has proceeded to zero. The audio output system comprises devices such as, but not limited to, buzzers, loudspeakers, bells, and the like. Furthermore, the timer is designed so that the audio output is transmitted acoustically to the tubing so that the user is able to hear the audio output directly through the earpiece of the stethoscope. The audio output system, or device, optionally signals the beginning of a countdown interval as well as the end of the countdown interval. The timer optionally further comprises a visual output device such as one or more light emitting diode (LED) to visually indicate the event of a countdown having proceeded to zero. The visual output device is optionally a device such as, but not limited to, a liquid crystal display, an active matrix alphanumeric or graphic display, a plasma display, or any of the standard graphic or alphanumeric displays used in commercial electronic equipment. Typical resolutions range from 10 by 10 pixels to 640 by 480 pixels or higher. The visual output device may further comprise a backlight to allow visualization in low light conditions when a reflective display, for example an LCD display, is used. The visual output device optionally signals the beginning of the countdown event as well as the termination of the countdown event. Either or both of the audio or visual output devices are optionally used to signal system status such as battery level, measured heart rate, and the like. The timer is preferably an electronic device that employs an electrical power supply such as a battery but the timer may also be a simple mechanical timer with a spring-loaded countdown clock and a mechanical lever or knob to “cock” the spring. The mechanical embodiment of the timer comprises one or more buttons or levers to begin the countdown event. A bell or buzzer signals the end of the countdown sequence. The timer, in the electrical embodiment, comprises one or more buttons or switches to initiate countdown events. Each button, preferably initiates a countdown event of different duration. In another embodiment, a single button may be depressed multiple times to achieve different countdown intervals. In an embodiment where the button is depressed multiple times, for example, the button is depressed once to achieve a six second interval, twice to achieve a 10 second interval, and three times to achieve a 20 second interval. The preferred countdown time intervals are 6 seconds, 15 seconds, and 30 seconds, however other countdown time intervals may be advantageously employed. In a preferred embodiment, the multiplication factor to convert the number of measured beats to beats per minute is labeled on the timer next to the button.

The timer further comprises connectors to permit the timer to be attached to a stethoscope. The connectors include devices such as, but not limited to, Velcro, clamps, clips, buttons, snaps, hooks, straps, and the like. The timer is, preferably, removably attached to the stethoscope so that the timer can be used with an already purchased stethoscope or is able to be switched from one stethoscope to another, a cost-saving benefit. The timer is, preferably, removably attached to the stethoscope at or near the end of the stethoscope comprising the auscultation head. A region proximate to the manifold or “Y” connector is also a preferable location for the timer. The timer is generally connected to the stethoscope between the auscultation head and the manifold. This placement allows for easy access of the timer while holding the bell shaped head against the patient to listen for heart or vascular sounds.

The stethoscope may be either a standard acoustic stethoscope, it may be an electronic stethoscope employing microphones, amplifiers, headphones, etc., or it may use a combination or hybrid of the aforementioned technologies. In continuing discussion, a human or any animal shall, at times, be covered by the term patient.

The stethoscope generally comprises one or more earpieces, a length of interconnecting tubing, and a sound receiver. The sound receiver may be referred to as the auscultation head or bell and is generally configured as a tapered or flared bell-shaped structure, the widest part of which is placed against the body. The auscultation head is generally affixed so its hollow central area is in communication with a hollow axially elongate tube. This axially elongate tube carries the sound received by the auscultation head to an earpiece. The earpiece is generally configured to fit into or around the ears and channel the sounds received by the auscultation head into the ears of a medical caregiver. The interconnecting tubing, in a preferred embodiment, further comprises a “Y” shaped manifold, “Y” connector, or “Y”, that splits the sound coming through a single interconnecting tube leading to the auscultation head. The manifold preferably splits the sound into two components and is audibly connected to interconnect tubing leading to an earpiece in each ear of the medical caregiver. Output channels beyond the normal two, three or four for example, are useful so that more than one person can hear the sounds generated by the stethoscope.

In the case of an electronic stethoscope, the auscultation head is replaced by a microphone. The earpiece is replaced by a loudspeaker or headphone. The interconnect tubing is replaced by wires or a wireless transmission receiver subsystem. An amplifier and signal processor further comprises the system to process the sounds from the microphone and amplify them for use by the loudspeaker or headphone system.

A primary aspect of the invention is the method of measuring the heart rate or breathing rate of the patient. A timer is affixed to a stethoscope, preferably near the end of the stethoscope that receives the cardiac or arterial pulse sounds. The medical caregiver listens to heart sounds or vascular pulse sounds. The medical caregiver depresses a button or switch on the timer that begins a countdown sequence of pre-determined length. When the countdown has completed or proceeded to zero, the medical caregiver counts the number of pulses heard during the countdown period. The medical caregiver then multiplies the number of pulses counted by the correct multiple to determine the number of beats per minute. In the case of a six second timer, the caregiver multiplies the number of pulses by ten to get the number of beats per minute. In the case of a fifteen second timer, the medical caregiver multiplies the number of pulses by four to get the number of beats per minute. In the case of a 30 second timer, the medical caregiver multiplies the number of pulses by two to get the number of beats per minute.

In another embodiment, the timer comprises input devices such as, but not limited to, buttons, switches, internal inertial switch, magnetic sensor such as a Hall effect sensor, a accelerometer, a gyroscope, and the like. The medical caregiver taps the input device or timer case in time or in synchrony with the heart rate as heard through the stethoscope. The timer comprises circuitry that calculates the heart rate in beats per minute. The heart rate is either calculated after a prescribed number of taps have been input or when the tap rate appears to be stable for a prescribed amount of time. The heart rate or breathing rate is displayed on a visual output device or audibly stated by an audio output device.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 illustrates an oblique view of a timer showing a visual output device, according to an embodiment of the invention;

FIG. 2 illustrates an oblique view of a stethoscope with a timer attached, according to an embodiment of the invention;

FIG. 3 illustrates an oblique view of a stethoscope timer showing an audio output device, according to an embodiment of the invention;

FIG. 4 illustrates a block diagram of a stethoscope timer, according to an embodiment of the invention; and

FIG. 5 illustrates an oblique view of a stethoscope timer comprising input devices to allow a medical caregiver to time the heart pulses, according to an embodiment of the invention.

### DETAILED DESCRIPTION OF THE INVENTION

In accordance with one or more embodiments of the present invention, a stethoscope, stethoscope timer and accessory components are described herein. In order to fully specify this preferred design, various embodiment specific details are set forth, such as the number and makeup of the countdown intervals, activation mechanisms, output devices, and the like. It should be understood, however that these details are provided only to illustrate the presented embodiments, and are not intended to limit the scope of the present invention.

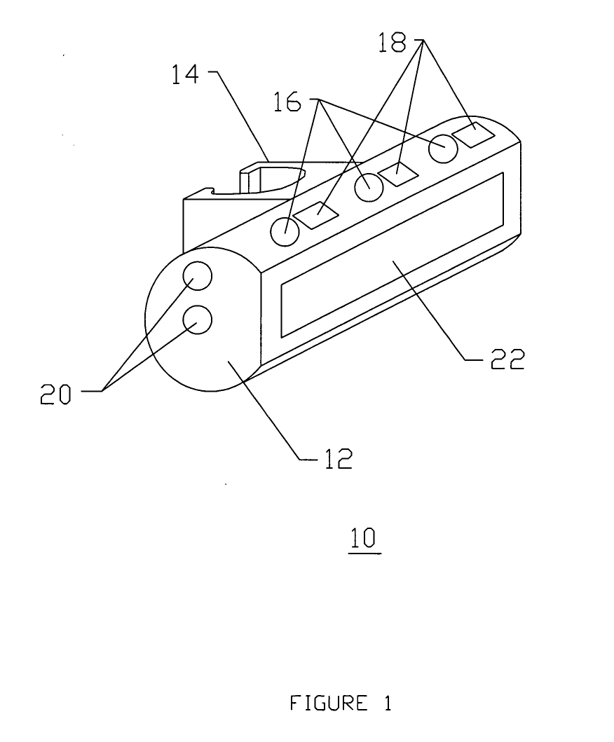

FIG. 1 illustrates an oblique view of a stethoscope timer 10 of the present invention. The stethoscope timer 10 comprises a case 12, one or more attachment clips 14, a plurality of countdown sequence start buttons 16, a plurality of countdown start button labels 18, one or more visual output devices 20, an advertising logo 22, a power supply 24 (not shown), and a timer circuit 100 (not shown).

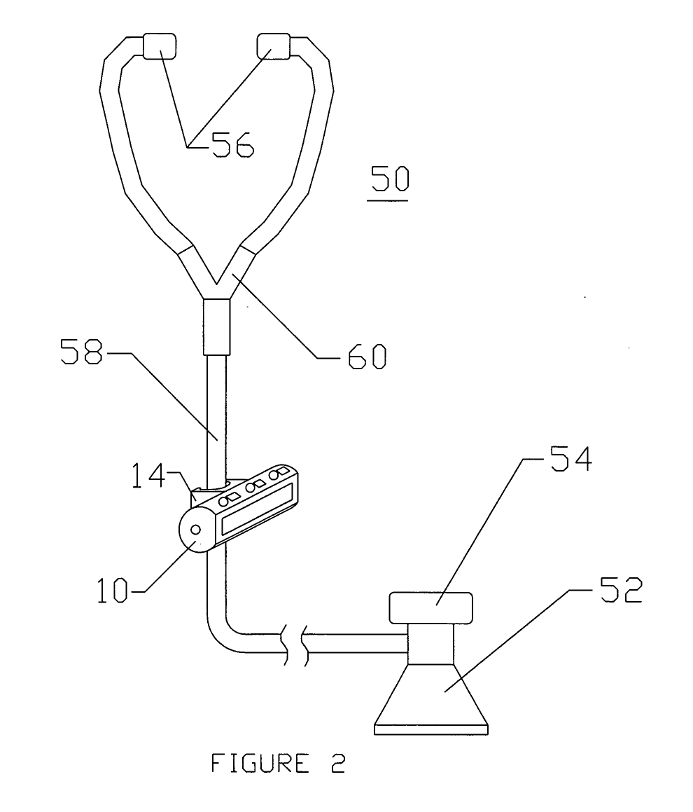

The attachment clips 14 are affixed to the outside of the case 12. The countdown sequence start buttons 16 are affixed to the exterior of the case 12 as are the visual output devices 20 and the advertising logo 22. The power supply 24 and the timer circuit 26 are affixed to the interior of the case 12. The power supply 24 preferably comprises a battery, which is preferably removable. In another embodiment, the battery is replaceable. In yet another embodiment, the battery is sealed to preclude replacement. Referring to FIGS. 1 and 2, the attachment clips 14 may run parallel to or perpendicular to the case 12 such that the timer 10 is aligned either parallel to, or perpendicular to, the tubing 58 of the stethoscope 50.

The attachment clips 14 are, preferably, permanently affixed to the case 12 but attachable or removable from a stethoscope. The clips 14 comprise structures such as, but not limited to, Velcro, snaps, buttons, spring-loaded jaws, setscrews, bayonet mounts, zippers, and the like. Referring to FIGS. 1 and 2, the clips 14 preferably are able to maintain an orientation of the timer 10 relative to the stethoscope 50, for example parallel to or perpendicular to the tubing 58, so that the timer 10 does not flop or rotate to a position in which it is difficult to operate the controls. In one embodiment, the timer 10 advantageously comprises a plurality of clips 14 to maintain orientation and secure the timer 10 to the stethoscope. Maintenance of rotational orientation is accomplished with the use of multiple clips 14 or by serrations or jaws on the clip 14 to prevent rotational slippage between the timer 10 and the stethoscope.

The case 12 is preferably rectangular in shape but may be advantageously fabricated in any geometry such as a triangle, circle, cylinder, cylinder or circle with a flat on one or more side, or any other polyhedral shape. The case 12, in one embodiment, is shaped to model a logo that is associated with a product, service, or company. The case 12 is lightweight so it does not strain the stethoscope or the neck of the caregiver. The case 12 is sized to be less than 6 inches in its largest dimension and less than 1 inch thick. The case 12 is preferably sized to be less than 3 inches in its largest dimension and less than ½ inch in thickness. In one embodiment, the case 12 is most preferably the approximate size of a small cylindrical container, such as the size of a lipstick container, a AA battery, or a “Chap Stick” container.

Referring to FIG. 1, the materials used in the manufacture of the timer case 12 or clip 14 include but are not limited to polymers such as polyvinyl chloride, PEBAX, acrilonitrile, butadiene styrene, PETG, PET copolymers, polyurethane, polyester, polyethylene, PEEK, polypropylene, polytetrafluoroethylene, polyetheretherketone, fluorinated ethylene propylene, polytetrafluoroethylene-perfluoromethylvinylether, and silicone rubber. The case 12 and the clip 14 may also be advantageously fabricated from metals such as, but not limited to, stainless steel, titanium, aluminum, anodized aluminum, brass, nitinol, and the like. The case 12 and the clip 14 are preferably injection molded but can also be made by techniques such as, but not limited to, CNC machining, laser machining, electron discharge machining, and the like. The case 12 is preferably ruggedized by the addition of a coating (not shown) of an elastomeric material such as, but not limited to, polyurethane, silicone rubber, latex rubber, and the like. Further ruggedization is accomplished internally by providing shock absorption to the internal components of the case 12 and by strengthening the electrical connections, therein, against fatigue and impact.

The countdown sequence start buttons 16 are preferably of the type that have a relative motion perpendicular to the plane of the case 12 on which the buttons 16 are mounted or affixed. The countdown sequence start buttons 16 are preferably waterproof and sealed against moisture exposure from the outside of the case 12. The countdown sequence start buttons 16 may also be advantageously of many types, including but not limited to, the membrane type, the capacitance type, knife switches, toggle switches, rocker switches, voice operated switches, inertial switches, or any other style of button or activator.

The countdown sequence start buttons 16 are associated with countdown sequence start button labels 18 that indicate the duration of the countdown sequence. The countdown sequence start button labels 18 further advantageously comprise information on the multiplier to be used to convert the number of beats measured to units of beats per minute. The countdown sequence start button labels 18 are located proximate to the countdown sequence start button 16 to which the label 18 is associated. The labels 18 are located so as to be unambiguously associated with the correct countdown sequence start button 16. The labels 18 are comprised of printed, lithographed, or pad printed material such as paper, coated paper, plastic, metals, and the like. The labels 18, in another embodiment are raised or embossed alphanumeric characters. The alphanumeric characters 18 are further distinguished by optionally tipping them with a hot stamp color or by pad printing, lithography, or the like.

The visual output devices 20 are comprised of light emitting diodes in a preferred embodiment. The visual output devices may further comprise devices such as, but not limited to, LCD displays, active matrix displays, light bulbs, and the like. More than one visual output device 20 is advantageously used to communicate system status to the user. In the simplest embodiment, the visual output device 20 blinks when the countdown sequence has reached zero. In a more sophisticated embodiment, the visual output device 20 illuminates with one color, green for example, when the sequence starts and with another color, red for example, when the sequence ends. In yet another embodiment, the visual output device 20 flashes at different rates when it is timing the countdown sequence from when the countdown sequence is completed. The flashing rates may vary from 0 to rates as high as approximately 100 Hz, or higher. In one embodiment, the visual output device 20 is on continuously. The visual output device 20 in yet another embodiment indicates the status of the battery and whether or not it requires replacement. In yet another embodiment, the visual output device 20 displays alphanumeric information relating to parameters such as, but not limited to, the measured heart or breathing rate, the beat multiplier, the battery status, the length of the timing interval, and the like.

The advertising logo 22 is a primary feature of the stethoscope timer 10. The advertising logo 22 is a label affixed to the case 12. The advertising logo 22 is pad printed, printed, lithographed, holographically printed, etched, embossed, molded in with raised letters, and the like. The advertising logo 22 is fabricated from materials and inks that are either coated or impervious to water, cleaning agents and any other environments to which the stethoscope timer 10 will become exposed in the medical environment. The advertising logo 22 is further protected or coated against abrasion and other forces to which it may be exposed in the hospital environment. In a preferred embodiment, the advertising logo 22 is a plurality of raised alphanumeric letters that are protected by raised edges or lips that minimize abrasive effects.

The power supply 24 preferably comprises a battery. The battery or set of batteries are standard easily replaceable cells such as those fabricated from chemistries such as, but not limited to, alkaline, lithium, nickel metal hydride, lead acid, and the like. The batteries may be non-rechargeable or they may be rechargeable using a plug attached to the timer 10 or by placing the timer 10 near a charger that comprises a coil capable of inducing a field within the timer 10 that charges the battery. Small flat batteries such as those used in watches are appropriate as are batteries such as AA or AAA size batteries sold commercially. The power supply 24 is preferably able to provide voltages to the timer 10 ranging from 1.2 to 12 volts and more preferably between 1.2 and 3 volts.

The timer circuit 100 receives its power from the power supply 24 and inputs from the countdown sequence start switches 16 as well as optional on-off switches and the like. The timer circuit 100 may further receive inputs from wireless sources such as, but not limited to, those generated by microwave, radio waves, ultrasound, infrared, and the like.

FIG. 2 illustrates a stethoscope 50 with the timer 10 attached. The stethoscope 50 comprises an auscultation head 52 further comprising a grip handle 54, one or more earpieces 56, an interconnection tubing set 58, and a manifold 60. The stethoscope timer 10 further comprises one or more clips 14.

The stethoscope timer 10 is preferably removably affixed to the stethoscope 50 by way of the clip 14. In this embodiment, the clip 14 comprises a plurality of grips that apply inward force to grip a tubular or cylindrical structure. The interconnection tubing set 58 comprises a tubular structure and the clip 14 attaches to the interconnection tubing set 58 with sufficient friction so that the timer 10 does not slide along the length of the interconnection tubing set 58 or rotate around the tubing set 58. The internal surfaces of the clip 14 that act against the stethoscope interconnection tubing set 58 comprise serrations, soft high-friction materials or the like to prevent or minimize slippage and rotation. Optional tabs (not shown) on the clip 14 permit the clip 14 to be opened and removed from the stethoscope 50. The timer 10 is positioned so that the medical caregiver can grasp the auscultation head 52 on the stethoscope 50 by way of the grip handle 54. Referring to FIG. 1 and FIG. 2, using the same hand or the other hand, the medical caregiver can grasp the timer 10 and operate the countdown start buttons 16 while listening to heart, chest, or cardiovascular sounds. In another embodiment, the timer 10 is affixed to the manifold 60 of the stethoscope 50. In yet another embodiment, the timer 10 is affixed to or proximate to the auscultation head 52. The timer 10 is preferably affixed to the stethoscope 50 between the auscultation head 52 and the manifold 60.

Referring to FIGS. 1 and 2, in one embodiment, the case 12 of the stethoscope timer 10 is oriented perpendicular to the axis of the interconnection tubing set 58. In another embodiment, the case 12 of the stethoscope timer 10 is oriented parallel to the axis of the interconnection tubing set 58. In yet another embodiment, a swivel joint that is optionally lockable is provided between the clip 14 and the case 12. This swivel joint permits the medical caregiver the opportunity to orient the stethoscope timer 10 at any preferred orientation relative to the stethoscope 50.

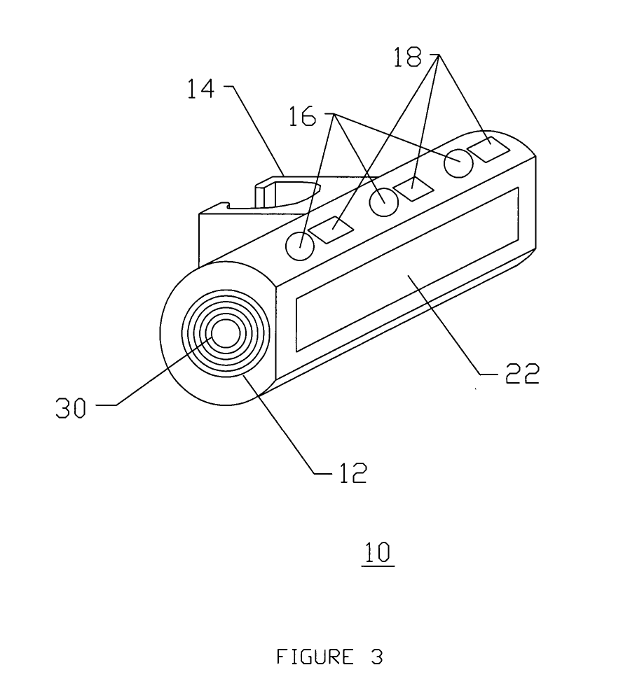

FIG. 3 illustrates an oblique view of a stethoscope timer 10 of the present invention. The stethoscope timer 10 comprises the case 12, one or more attachment clips 14, the plurality of countdown sequence start buttons 16, the plurality of countdown start button labels 18, an audio output device 30, the advertising logo 22, the power supply 24 (not shown), and the timer circuit 26 (not shown).

This embodiment differs from the embodiment in FIG. 1 in that it comprises the audio output device 30. The audio output device 30 comprises a loudspeaker, buzzer, beeper, alarm, or similar device to generate audio frequencies that are audible to the human ear. The audio output device 30 is affixed to the case 12 of the timer 10. In one embodiment, the audio output device 30 is preferably affixed to the interior of the case 12, which is further perforated to permit audio sound waves to escape the environs of the case 12. The frequency range of the audio output device 30 is preferably such that a person who is hearing impaired can still hear the device. Thus, the frequency range is between approximately 100 Hz and approximately 10,000 Hz, but preferably between 150 Hz and 4,000 Hz. In a preferred embodiment, the audio output device is electrically driven by an audio amplifier and a frequency generator or logic circuit, further comprising a digital to analog converter, which are powered by the power supply 24. In an embodiment, the audio output device 30 is rigidly affixed to the case 12, which is further affixed to the stethoscope tubing in such a way that the audio output is acoustically transmitted to the stethoscope tubing. In this embodiment, the user is able to hear the output of the audio output device 30 directly through the earpiece of the stethoscope via acoustic transmission in the stethoscope tubing. The audio output is in the form of pulses of sound or it is in the form of recognizable language, preferably tailored to the country of use. For instance, the audio output is, in one embodiment, English language and comprises words such as, but not limited to, “Timer Start”, “Countdown Start”, “Countdown End”, “Heart Rate 52 Beats Per Minute”, “Battery Level Low”, and the like. Referring to FIGS. 1 and 3, in another embodiment, the timer 10 advantageously comprises both an audio output device 30 and a visual output device 20.

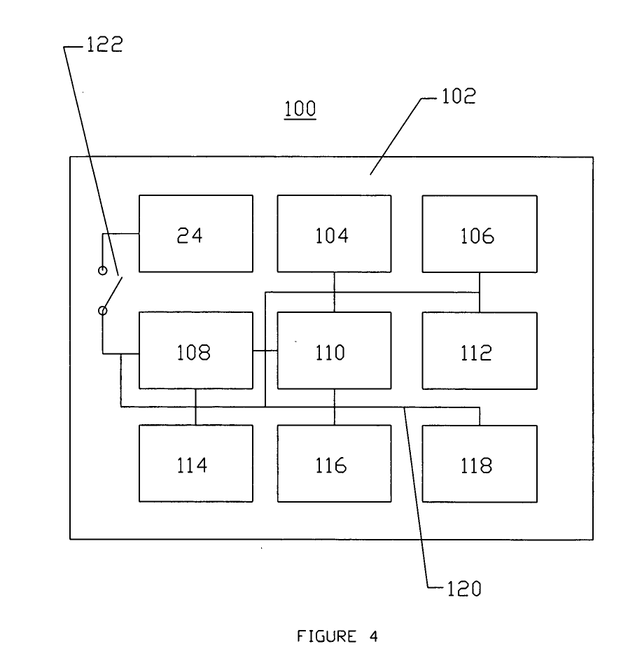

FIG. 4 illustrates a block diagram of a stethoscope timer electrical subsystem 100. The electrical subsystem 100 comprises a chassis or circuit card 102, a power supply 24, an on-off switch 122, a relay 104, an audio output device 106, a clock 108, a logic circuit 110, an optional visual output device 112, one or more countdown start switches 114, an optional alphanumeric display 116, and a rate input device 118, and an electrical bus 120.

The electrical subsystem 100 of the timer 10 is housed within the case 12 of the timer 10. The electrical subsystem 100 preferably comprises a chassis or circuit card 102 to which all components 24 and 104 to 122, are mechanically connected. The components are electrically interconnected by the electrical bus 120. In an embodiment, when the countdown start switch 114 is depressed, the countdown begins immediately. In another embodiment, the clock 108 and logic circuit 110 introduces a delay of between 1 and 20 seconds, and preferably between 1.5 and 5 seconds, before the countdown sequence begins. This allows the user to place the stethoscope on the patient before the countdown sequence begins.

The power supply 24 is preferably a battery such as that described in FIG. 1. The power supply 24 is preferably removably affixed to the electrical subsystem 100, but may be advantageously non-removable in another embodiment. The power supply 24 supplies power to the electrical bus 120 of the electrical subsystem 100 and is preferably switched by an optional on-off switch 122. The on-off switch is a manual switch such as a pushbutton or a rocker switch. Referring to FIGS. 1 and 4, the on-off switch 122, in a preferred embodiment, is an automatic switch that turns on when the start switch 114, such as the countdown sequence button 16, is depressed or when the timer 10 detects motion such as with a motion-detecting device. Such a motion-detecting device is, for example, a gyroscope, accelerometer, inertial switch, or the like.

Referring to FIGS. 1, 2, and 4, the clock 108 is a standard commercial clock device such as that used with computers and other logic devices. The logic circuit 110 is a conventional computer and need not be highly sophisticated. A standard 8-bit controller device is appropriate for this application. The logic circuit 110 comprises appropriate memory, either random access memory (RAM) or permanent memory such as ROM or EPROM, or both. The start switch 114, when enabled, inputs the start of a countdown event. The relay 104, takes information from the logic circuit 110 and sends power or other information to the audio output device 106, and/or the visual output device 112. The alphanumeric display 116 is driven by a video controller (not shown), based on information output by the logic circuit 110. The rate input device 118 is, in one embodiment, a simple switch. The rate input device 118, in a preferred embodiment, is an inertial device such a switch mounted or affixed to a trampoline or other elastomeric surface. An inertial mass is also affixed to the elastomeric surface such that motion of the case 12 of the stethoscope timer 10, causes the inertial mass to move relative to the case 12. This motion of the inertial mass causes motion in a magnetic sensor, an electromagnetic sensor, an electrical field switch, or simple electrical contact, a capacitance change, a resistance change, or the like. By tapping on the case 12 of the stethoscope timer 10, the rate input device 118 sends pulses to the logic circuit 110, which calculates a rate based on averaging the input pulses. The tapping on the case 12 is done by the medical caregiver in synchrony, or approximate synchrony, with the audible physiological sounds heard through the stethoscope 50. The determined rate (heart rate or breathing rate) is then output on the alphanumeric display 116 or audibly output on the audio output device 106.

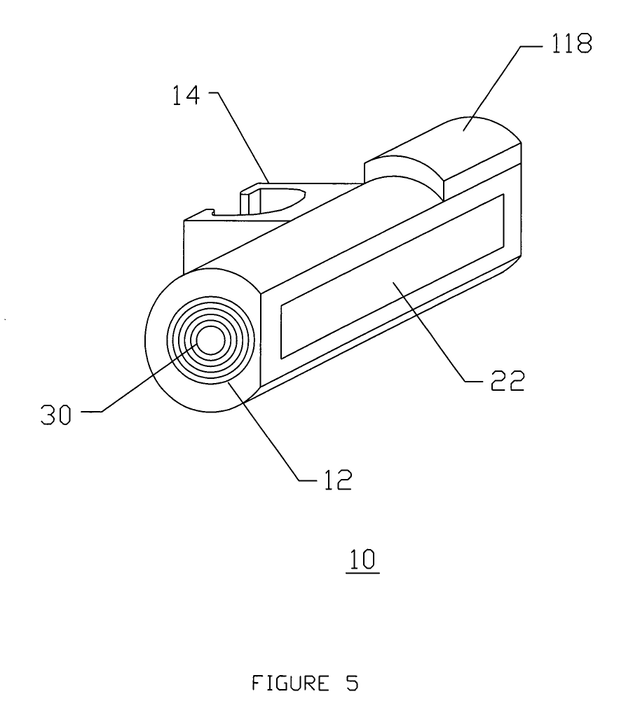

FIG. 5 illustrates a stethoscope timer 10 that further comprises a rate input device 118 and electronic circuitry (not shown) to calculate and display the heart or breathing rate. Manual tapping on the case 12 or depression of a rate input device 70, which is a button in this instance, on the stethoscope timer 10 in general synchronization with the audibly detected (through the stethoscope 50 of FIG. 3) heartbeat generates an input of the heart or pulmonary rate.

The rate input device 118 is, in this embodiment, a switch that is depressed in time with cardiac or pulmonary sounds. The rate input device 118 is affixed to the case 12 of the stethoscope timer 10. The rate input device 118 does not prevent clear view of the advertising logo 22. The preferred output device is the audio output device 30, as shown in this embodiment.

Referring to FIGS. 1 and 2, major feature of the invention is the method of promotion or advertising of a medical product or service. The name or other identifying logo of the provider of the medical product or service, or the name of the product or service is affixed to an advertising logo 22 that is further affixed to the case 12 of the stethoscope timer 10. This provides a business model to gain notoriety for the company, product, or service. The stethoscope timer 10 is a promotional device that is given or sold to medical caregivers for use with their stethoscopes. The stethoscope timer 10 is attachable to any standard stethoscope by way of the clip 14. This promotional device further permits easier and more accurate measurement of heart or breathing rates and thus facilitates the practice of medicine.

Referring to FIG. 2, in yet another embodiment, the stethoscope timer 10 is configured to communicate with an electrical stethoscope 50 and directly detect the heart rate based on pulses measured by the stethoscope and transmitted to the stethoscope timer 10 by electrical wire or wireless means such as Bluetooth technology, infrared, microwave, ultrasound, RF, or the like. Optionally, the stethoscope timer 10 comprises a coil or detector that can receive, or steal, electromagnetic information from the stethoscope 50 or the wires running through the stethoscope 50. In this embodiment, the weak electromagnetic signals are amplified and processed to decode the heart or breathing rate information within the stethoscope timer 10. The stethoscope timer 10 audibly outputs the heart or breathing rate or provides a visual output of the heart or breathing rate.

Referring to FIG. 2, in yet another embodiment, the stethoscope timer 10 is configured to affix to an acoustic stethoscope 50 by way of the clip 14. The clip 14, in this embodiment, preferably attaches to the interconnection tubing set 58 of the stethoscope 50. The clip 14 comprises sensitive microphone or pressure sensors capable of detecting, or stealing, the sound waves within the stethoscope 50. The microphone or pressure sensors feed signal processors and amplifiers that provide input to an analog to digital converter that further provides input to the logic controller. The stethoscope timer 10 audibly outputs the heart or breathing rate or provides a visual output of the heart or breathing rate.

The advantage of the aforementioned devices and methods improves the ease with which a heart rate or pulse may be measured, especially by less well-trained personnel such as paramedics and emergency medical technicians.

Application of the stethoscope timer permits a medical caregiver to easily take a pulse rate in the manner to which they are accustomed but without needing to look away at a clock. The application of this removable timer allows retrofitting of standard stethoscopes with a work-saving and accuracy improving aid to obtaining a heart rate or a breathing rate.

The present invention may be embodied in other specific forms without departing from its spirit or essential characteristics. For example, the stethoscope timer may or may not include a logo or advertisement and the number and duration of the countdown intervals may vary. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. An apparatus adapted to measure the heart rate of an animal comprising:
a stethoscope further comprising an auscultation head, one or more earpieces, and interconnecting audio information channels; and
a timer affixed to the stethoscope;
wherein the timer is a countdown timer.

2. The apparatus of claim 1 wherein said timer is removably affixed to the stethoscope.

3. The apparatus of claim 1 wherein said timer is removably affixed to the stethoscope at or near the auscultation head.

4. The apparatus of claim 1 wherein said timer further comprises an advertising logo or message.

5. The apparatus of claim 1 wherein said timer comprises one or more buttons to initiate the timer countdown.

6. The apparatus of claim 1 wherein said timer further comprises an audible signal to indicate that the countdown has processed to zero.

7. The apparatus of claim 1 wherein said timer further comprises a visual output device to indicate that the countdown has processed to zero.

8. The apparatus of claim 1 wherein said timer further comprises a touch input device that is activated manually by the user in synchronization with the heart beat.

9. The apparatus of claim 1 wherein said timer further comprises a visual output display of the heart rate.

10. The apparatus of claim 1 wherein said auscultation head is a microphone and said ear piece is an audio output device, the stethoscope further comprising electrical interconnects between the auscultation head and said earpiece, as well as amplification and power supply subsystems.

11. The apparatus of claim 1 wherein said auscultation head is an acoustic receiver and is interconnected to said earpiece by hollow sound wave transmitting tubing.

12. The apparatus of claim 1 further comprising a “Y” manifold to divide the acoustic or electrical output of the auscultation head to permit more than one ear to listen to said electrical or acoustic output.

13. The apparatus of claim 6 wherein said audible output is different depending on the time selected for countdown.

14. The apparatus of claim 7 wherein said visual output is different depending on the time selected for countdown.

15. A method of measuring the heart rate on a human patient or other animal that involves the steps of:
affixing a timer to a stethoscope;
listening to the sounds of the heart or vasculature;
starting the countdown timer attached to the stethoscope;
counting cardiovascular pulses based on the sounds of the heart or vasculature;
discontinuing counting upon completion of a countdown event generated by the timer; and
determining the heart rate based on the number of cardiovascular pulses measured until the completion of the countdown event and the time interval used for the countdown event.

16. The method of claim 15 wherein the time interval is determined by depressing a discrete button to activate the countdown for that pre-determined time interval.

17. The method of claim 15 wherein the completion of the countdown event is signaled by one or more audible beeps generated by the timer.

18. The method of claim 15 wherein the completion of the countdown event is signaled by one or more pulses of light generated by the timer.

19. The method of claim 15 further comprising tapping on the timer in general synchronization with the heart beat to count pulses.

20. The method of claim 15 further comprising the step of using the timer to advertise a logo or information about a company, product, or service.
